(19) United States
(12) Patent Application Publication
(10) Pub. No.: US 2025/0010101 A1
(43) Pub. Date: Jan. 9, 2025

(54) MCO SELECTION OF TREATMENT TECHNOLOGIES IN RADIOTHERAPY METHOD, TECHNICALLY FUNCTIONAL GUI, AND USE

(71) Applicant: FRAUNHOFER-GESELLSCHAFT ZUR FOERDERUNG DER ANGEWANDTEN FORSCHUNG E.V., Muenchen (DE)

(72) Inventors: Michael BORTZ, Kaiserslautern (DE); Karl-Heinz KUEFER, Weilerbach (DE); Katrin TEICHERT, Kaiserslautern (DE); Philipp SUESS, Budenheim (DE)

(21) Appl. No.: 18/558,516

(22) PCT Filed: May 3, 2022
(86) PCT No.: PCT/IB2022/054081

§ 371 Date: Aug. 23, 2024

(30) Foreign Application Priority Data
May 3, 2021 (DE) 10 2021 111 420.9
Sep. 3, 2021 (DE) 10 2021 122 848.4

(51) Int. Cl. A61N 5/10

(52) U.S. Cl. CPC A61N 5/1031; A61N 5/1048; A61N 2005/1074; A61N 2005/1087

(57) ABSTRACT

The aim of the invention is to allow patients to be individually provided with good therapy, whereby the quality of the therapy also includes minimizing the waiting time of each patient (under “time to treatment”). Proposed is a method for designing or fashioning a therapy as a treatment plan, before treatment, and with interactive navigation on a display device (10). Multiple technologies (A, B, . . . Z) of radiation devices (100, 200, 300) are available for selection, including at least one technology (A) with a radiation device (100) for emitting protons and at least one technology (B) with a radiation device (200) for emitting photons. After designing or fashioning, for a person (P) who is to be treated with therapy that can be provided by the radiation device (100, 200, 300), the designed or fashioned plan defines a plurality of technical settings that are adjusted on the radiation device(s) of the selected technology/technologies. For at least some of the technologies (A, B, . . . Z), one Pareto frontier each (101, 201, 301) is shown as a patch in an interactive patch area on the display device (10). An operating area on the display device (10) shows a number of operating aids (21, 22, . . . ), each of which represents a criterion (c1, c2, . . . ) that is improved when a selector (21a, 22a, . . . ) of the respective operating aid is moved or operated in one direction or worsened when it is moved or operated in the opposite direction. A fuzziness interval (30, 31, 32) is defined for a target criterion (c1, c2, . . . ), which is also displayed accordingly in the interactive patch area and covers at least two Pareto frontiers (201, 301) for at least two technologies (A, B). An input from the planner interactively conveys the equivalence of two technologies to the same planner. The two technologies shown (201, 301) have the same value (c1₁) for the same target criterion (c1) for the respective patch.

The present disclosure (and claims) relate to a method of multi-criteria optimization (MCO). Also affected is a GUI, either stand-alone or for application (use) in these methods. Seen in this way, the use is a new use of a function pointer known as such.

A cancer patient is to receive radiation therapy. This involves a minimum dose for the tumor tissue and maximum doses for surrounding healthy tissue structures, which should ideally not be exhausted. In principle, there are multiple technical options (technologies) with which such a treatment can be carried out for such a patient. Examples include the following:
1. Protons
2. Photons per rotation therapy with 1 rotation
3. Photons per rotation therapy with 2 rotations
4. Photons per IMRT with 9 fields (with 9 fixed angles)
5. Photons per IMRT with 9 fields (with angles other than those in option 4)
6. Other technologies

Each of these technologies can be performed on one or more different therapy devices. Each device is addressed differently and requires specific planning, whereby the individual planning objectives remain identical across all of the technologies used.

Also relevant from a clinical standpoint is the planning of all treatment cases (overall planning), taking into account all available treatment devices. Besides taking into account the individual medical aspects of a therapy, the overall planning also needs to schedule the treatments.

Mathematically, a multi-criteria planning problem arises with the following objectives: high probability of successful treatment while avoiding side effects with each individual therapy and making the most effective use of the available therapy devices, with which as many patients as possible can receive therapy without having to endure long waiting times.

The state of the art is a two-stage decision-making approach. To begin with, the technology is defined, and then this technology is planned. Multi-criteria planning methods can be used in the second step. Doctors decide on the choice of technology based on experience and the results of clinical studies. In order to decide which technology is best suited to a treatment case, it is necessary to undertake separate planning for each possible technology, compare the respective plans, and then select the “best” option.

The clinical procedure also needs to be considered: in urgent cases, treatments already scheduled on certain devices will have to be postponed or even rescheduled on other devices, or else the patient in urgent need of treatment will have to wait.

Each technology must be implemented on specific, dedicated devices—treatment with protons cannot be performed using a photon accelerator. The number of devices available for treatment is one of the limiting factors in hospitals. Treating more patients—proton systems in particular are only available to a very limited extent due to their cost and complexity—automatically leads to waiting times or rescheduling of therapy sessions. A multi-criteria optimization problem becomes apparent: treating as many patients as possible in a given time—or with a given, usually limited amount of investment capital.

The technical problem of the invention lies in the ability to individually provide each patient with good therapy, whereby the quality of the therapy also includes minimizing the waiting time of each patient (under “time to treatment,” a technical criterion of therapy planning).

The invention is based on the realization that, for individual patients, a proton treatment cannot be identified as being clearly better due to uncertainties described below and that the treatment case can also be solved with a variant of photon therapy. This allows radiation devices to be kept unoccupied for proton treatment in cases where this is indispensable, or treatments can be performed on a photon radiation device, for example, where a little more time is required.

The navigation of a Pareto frontier across patches (one patch corresponding to one technology) is supplemented by a fuzziness range, whereby a threshold value can indicate that, below its value of 10%, for example, two technologies—while not equal—are equivalent, meaning that the use of one for the therapy is just as suitable as the other.

The threshold value can be set using a separate operating aid, or it can be visibly set by changing the width of one (or both) selectors of the operating aids provided for navigating the criteria.

There can be as many operating aids as there are criteria to be navigated (target criteria or planning objectives).

The solution proposed is an initial method for designing or fashioning a therapy as a treatment plan, before treatment, and with interactive navigation on a display device. Multiple technologies are (visibly) available for selection, including at least one technology with a radiation device for emitting protons and at least one technology with a radiation device for emitting photons. Other radiation devices are specified in the dependent claims.

The designed or fashioned plan defines a plurality of technical settings that are adjusted on the radiation device(s) of the selected technology/technologies. For at least some of the technologies, one Pareto frontier each is shown on the display device. Each of these frontiers is called a patch. An operating area and a patch area are provided on the display device. A number of operating aids are shown in the operating area, each of them representing one criterion. When a selector of a respective operating aid is moved or operated, the criterion shown by this operating aid is improved in one direction or worsened in the opposite direction (to be read as “and” but not possible at the same time). At least some of the operating aids can be shown as sliders. In most cases, a movement to the left results in an improvement.

The selectors form (and enable) interactive communication with the planner. A solution selected in the current state is shown as a point (zᵢ) in the patch area on one of the multiple Pareto frontiers. This means that the patch area and the operating area are functionally linked. What is selected in the operating area is also displayed in the patch area. If the selected solution in the operating area is changed when (or while) the planner changes the position of a respective selector of a respective operating aid, the point (zᵢ) in the patch area moves along multiple Pareto frontiers and thus from one patch onto the following patch.

Navigation across multiple patches, and thus technologies, makes a technology border disappear or blur, meaning that that we can speak of simultaneous navigation.

A fuzziness interval is provided that is made visible in the patch area and in the operating area. It is defined for a target criterion, such as in the operating area. To this end, it is displayed in the interactive patch area. The fuzziness is described in such a way that it covers at least two Pareto frontiers (which stand for at least two technologies). An input from the planner uses the fuzziness to visually convey an equivalence of two technologies, whereby the two technologies shown have an equal value c1₁ for the same target criterion (e.g., c1) in all patches, but the fuzziness does not extend so far that non-equivalent technologies are reached.

Another target criterion shown in the operating area does not have the same values as the assumed value c1.

A resulting Pareto frontier may—but need not—be shown on the display device as a patch that tracks (or picks up) multiple sections of multiple Pareto frontiers already shown as patches of shown technologies to make it more visible or clear to the planner that they are navigating the resulting patch (801) across technologies to find a treatment plan. The resulting Pareto frontier can be a non-dominated set of a union of Pareto frontiers.

A combinatorial patch with first parts of the first patch and second parts of the second patch can be calculated from two shown patches and shown on the display device for the planner's navigation. Another patch is created on the display device, which is derived from parts of the shown patches. The planner can navigate on it in the same way as on the patches that depict the technologies or the resulting Pareto frontier. The latter does not need to be shown.

An alternative method permits the designing or fashioning of a treatment plan with interactive navigation on an input/display device. The device includes a display device. For at least some of the technologies, one Pareto frontier each is shown in a Pareto frontier diagram on the display device. Each of the Pareto frontiers is to be referred to as a patch. Each of the Pareto frontiers is displayed in the patch area of the display device depending on at least two criteria. A number of operating aids are shown in an operating area outside the patch area. These may be shown as sliders.

A planner can choose from multiple treatment technologies for the plan that is to be designed or fashioned. The designed or fashioned treatment plan defines a plurality of technical settings that are adjusted on the at least one radiation device of the selected technology/technologies.

For at least some of the technologies, one Pareto frontier each is shown in the Pareto frontier diagram. Each of the Pareto frontiers is displayed in the patch area depending on at least two criteria. The criteria are shown by operating aids. Each operating aid is assigned to a criterion—or vice versa. A selector is used to change a criterion. Moving or operating a selector of the respective operating aid in one direction improves the criterion, or moving or operating it in an opposite direction worsens the criterion.

Each operating aid uses a selector—for interactive communication with the planner—to show a solution selected in the current state in the patch area as a point zᵢ on one of the multiple Pareto frontiers. The selected solution is changed along multiple patches during interactive communication with the planner.

The aim is to provide a fuzziness interval defined for a target criterion. It is also displayed accordingly in the interactive patch area and covers at least two Pareto frontiers (as at least two treatment technologies). This allows an input from the planner to interactively convey the equivalence of two technologies to the same planner. The two treatment technologies shown have the same value (c1₁) for the same target criterion (c1) in the respective patch. The position of the selector of an operating aid is changed linearly (as a slider) or as a rotational position (rotational specification). The operating aids are depicted on the display device and operated using a mouse pointer or directly with a finger on a touch-enabled screen (touchscreen).

With the slider, the operating aid is linear. For an operating aid with a variable rotary position, the operating aid has a curved adjustment range. As a rule, the operating aids are depicted in two dimensions, but this does not limit their understanding.

More than two target criteria (including plan targets) can be shown on the display device by means of projections in the patch area. The depiction of two axes is a strong simplification that can certainly be extended into multiple dimensions. One type of representation is projection.

One of the target criteria can be treatment time. Another target criterion can be the number of fractions (of a treatment). Additional target criteria are also possible; the specified criterion is not a stand-alone criterion.

The fuzziness interval can be shown interactively by widening the selector of the operating aid.

It can also be represented in the patch area by an extended field as a window for interactive communication with the planner (the user of the tool). It includes at least two Pareto frontiers as at least two patches.

The window in which the planner marks—i.e., selects or deselects—technologies (including treatment technologies) can be displayed on the display device. The window can be designed in such a way that it can be operated by the user using a mouse pointer or touchscreen in order to highlight the technologies (or treatment technologies) displayed in it.

The window becomes a function field in the operating area. The technologies (or treatment technologies) shown with it (or in it) correspond to the Pareto frontiers, such as for selecting or deselecting by the planner. When selected, they are also displayed or shown in the operating aids. If the technology is not checked (or selected), it is not displayed as an operating element in the associated operating aid. The selection or deselection process can take place multiple times in succession.

A selected solution can be changed along the at least one patch by the planner changing the position of a respective selector of a respective operating aid. There can be several ways of changing the position of the selector, such as by changing the position of the point in the patch area or by changing the position of the selector of a respective operating aid in the operating area.

The patch area can thus become an operating area as well; in other words, it is also possible to operate from the patch area.

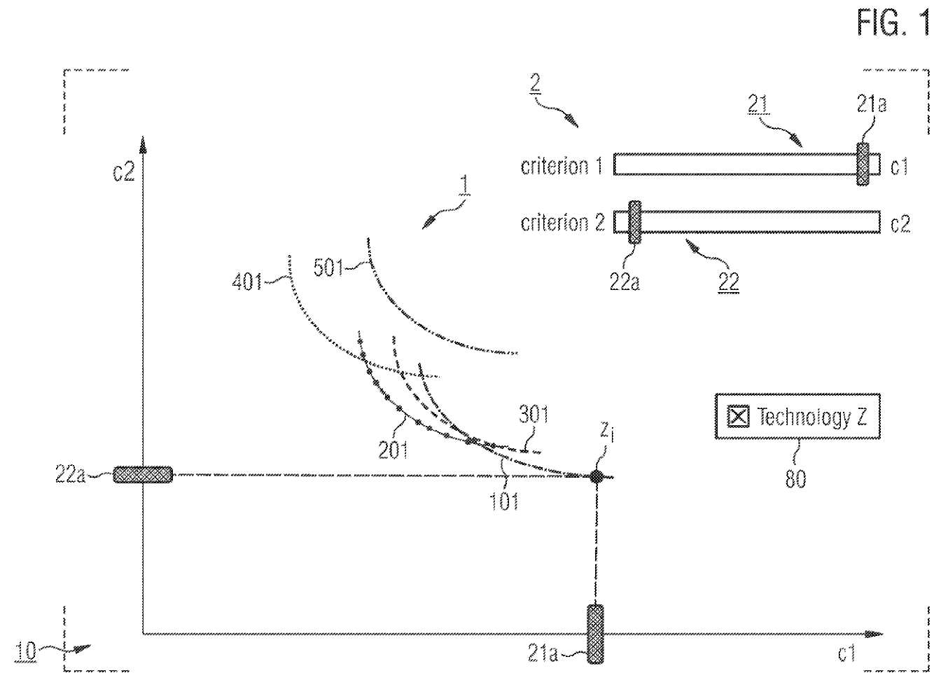

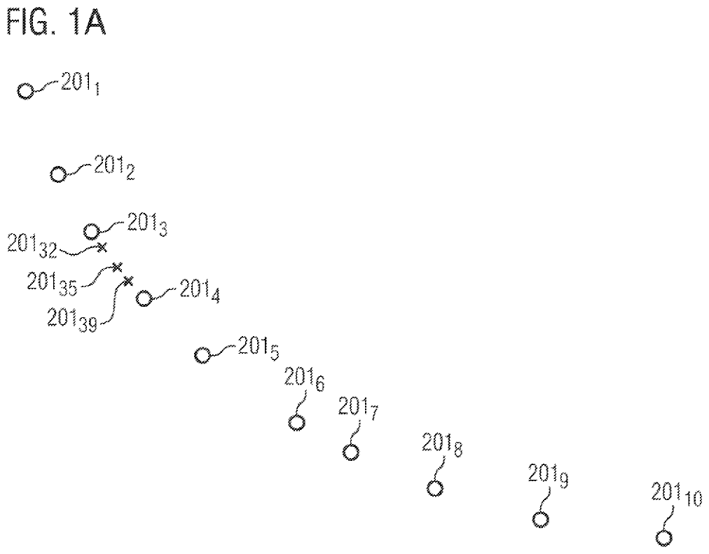

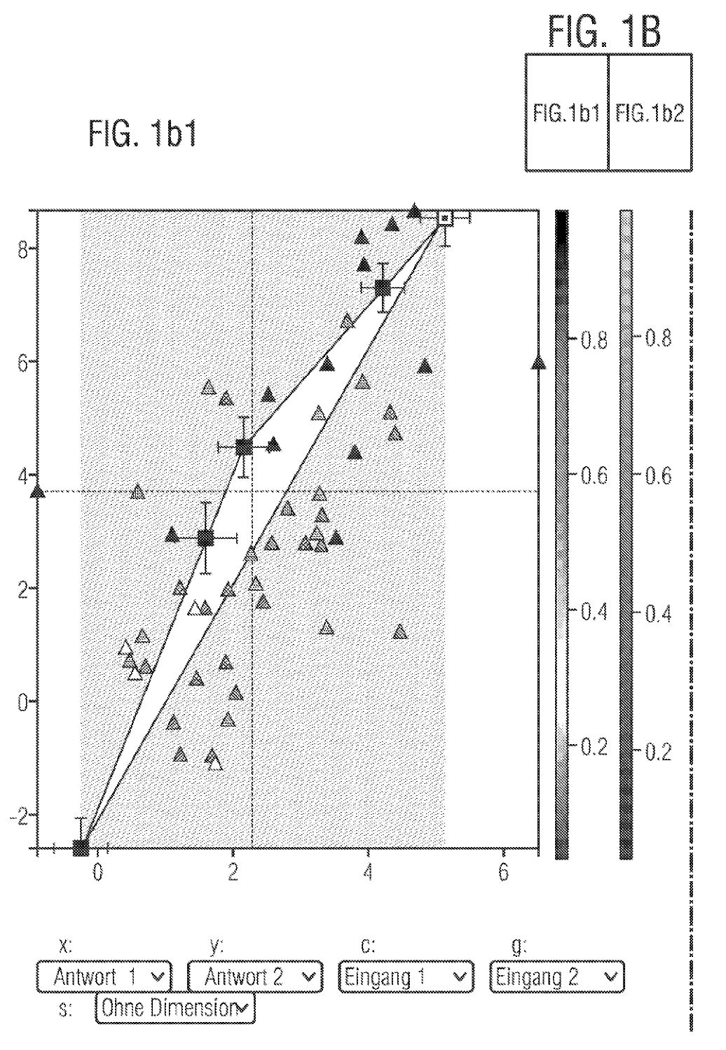

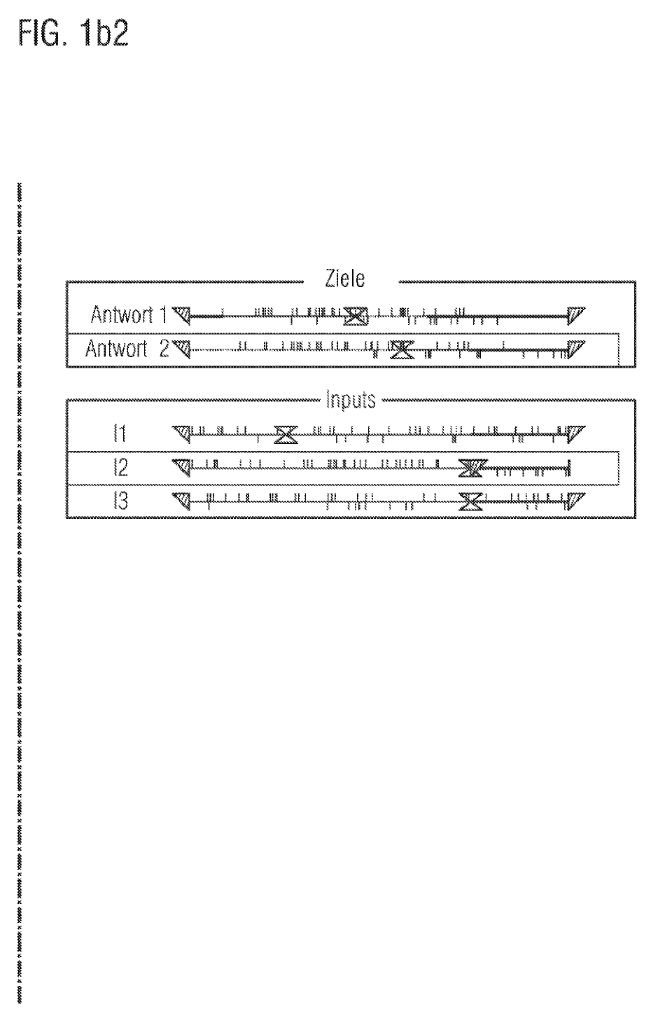

The following should be noted for understanding the Pareto frontier(s). Each Pareto frontier is a function that is only functionally connected. Each of them has a large number of grid points. The frontier is therefore by no means to be understood as continuous but only as “functionally connected;” therefore, a line is also shown in FIG. 1 for the grid points, which basically does not represent a continuous Pareto frontier. It can also be a projection from the n-dimensional space in the space in which it exists, whereas it can only be shown graphically as a projection. The lines represent the functionally connected Pareto frontier. This is illustrated in enlarged form in FIG. 1A, with interpolation between the grid points.

The Figures serve as examples to aid in understanding the general statements.

Reachable points on the multidimensional Pareto frontiers can be shown differently. The current position (the navigated point) is in the crosshairs, such as in FIG. 1b1. Reachable points can be shown in light or contrasting colors. Points that cannot be reached, such as a projected surface, have a different contrast or color.

Color coding that is not shown can be used to represent a dimension that is not visible yet still present. Uncertainties can be represented in five larger squares. A center of each of these would be the expected value; four arms in 90° orientations each indicate such areas of uncertainty as an interval.

Internal or external radiation devices can be used. The at least one radiation device involves at least one radiation device for emitting protons or photons, for emitting accelerated heavy ions, for emitting accelerated electrons, or it is a brachy device for emitting photons or gamma rays from (individually) advanced radiation bodies (subject to radioactive decay). These radiation bodies can have a cylindrical shape with a length of less than 5 mm and consist of cobalt-60, iodine-125, or iridium-192, preferably surrounded by a titanium shell. Other radioactive decaying substances are also possible.

A radiation device according to the claim is therefore also to be understood as a brachytherapy device, such as an afterloader. In the afterloading process, the source of radiation is inserted by a kind of robot under computer control. The patient lies alone in a radiation-proof room. This means that medical staff do not come into direct contact with the sources of radiation, previously specified as “radiation bodies.” This is due to their size, not their harmless nature.

Accordingly, the radiation device emits radiation, exposing the patient externally (radiotherapy) or internally (with inserted radioactive radiation bodies, i.e. emitting photons or gamma radiation).

Brachytherapy is used to treat cervical, prostate, breast, and skin cancer as LDR (low-dose-rate brachytherapy) at up to 2 Gy/h (Gray/h), MDR (medium-dose-rate brachytherapy) at an average dose rate of between 2 and 12 Gy/h), as HDR (high-dose-rate brachytherapy) at a dose rate above 12 Gy/h, or as PDR (pulsed-dose-rate brachytherapy) in which brief radiation pulses are emitted, such as once an hour, in order to mimic the overall intensity and effectiveness of LDR treatment.

Planning is required for all variants but not for the actual treatment.

With brachytherapy, the source of radiation is introduced into the (human) body in several steps. To begin with, one or more applicators are used. These aids for introducing one or more sources of radiation (the radiation bodies) can be thin plastic or metal tubes (catheters or cannulas), for example. X-rays, ultrasound, computer tomography, or magnetic resonance imaging (MRI) are used to check whether the applicators are in the right place in the body. If this is the case, the source(s) of radiation is/are introduced in a further step.

With brachytherapy, the source of radiation is therefore either in the immediate vicinity of the tumor (the target), or it is introduced directly into the target. The rays only travel a short distance in the body (ancient Greek “brachys” means “short,” “close,” from which the name of this therapy is derived).

The aim of radiotherapy for malignant targets is to destroy the tumor while sparing the neighboring healthy tissue as much as possible. The latter is a particular concern of brachytherapy, in which radioactive emitters are inserted directly into the target or its immediate surroundings in a minor procedure. Unlike with external radiation, the rays reach the target directly and thus cause less damage to the surrounding healthy tissue.

Historically, after an initial interest in brachytherapy in Europe and the US, its use declined in the middle of the 20th century because the radiation exposure for the treating doctors was too high when handling the sources of radiation manually. However, greater interest has currently re-emerged, also due to the development of remote-controlled reloading systems (called afterloading) and the use of new sources of radiation, which have reduced the risk of high radiation exposure for doctors and patients; for example, see claim 14. Advancing the radiation sources in the specified paths, such as catheters, guides them to the target; otherwise, the sources of radiation are retracted in a radiation safe to protect the environment, nurses, and doctors.

A combined, global Pareto frontier can be formed from partial areas of the multiple Pareto frontiers; preferably, the selected solution is changed along the combined global Pareto frontier.

Claim 16 is incorporated here by reference without reference signs.

Technical solutions to said technical problem are also found in claims 20 or 21, which claim the development of a graphical user interface (a GUI) and whose content is included here.

A first GUI works with a representation on a display device to execute the process. For at least some technologies, the GUI shows one Pareto frontier each in a Pareto frontier diagram on a display device of the input/output device. Each of the Pareto frontiers is referred to as a patch. Each patch is displayed in a patch area of the display device depending on at least two criteria. A number of operating aids are shown in an operating area on the display device outside the patch area, with each operating area representing one criterion. The operating aids each have one selector, whereby the moving or operating of a selector of the respective operating aid in one direction improves a corresponding criterion, or moving operating it in an opposite direction worsens the criterion. For interactive communication with the planner, each operating aid uses the associated selector to show a solution selected in the current state in the patch area as a point zᵢ on one of the multiple Pareto frontiers, whereby the selected solution is changeable (continuously) along multiple patches. A fuzziness interval can be determined (or variably defined) and defined at a target criterion, which, represented accordingly, is also displayed in the interactive patch area by a visually highlighted strip and covers at least two Pareto frontiers as at least two treatment technologies with its extension. This allows an input from the planner to interactively convey the equivalence of two technologies to the same planner in such a way that the two treatment technologies shown have the same value c1₁ of the same target criterion in the respective patch.

Another GUI is suitable for designing or fashioning a therapy as a treatment plan in an interactive navigation on a display device. For at least some of the technologies, one Pareto frontier each is shown connected on the display device, each of which is referred to as a patch. A number of operating aids are shown in an operating area on the display device, each of them representing one criterion that permits moving or operating a selector of each operating aid. For interactive communication with the planner, the respective selector highlights a solution selected in the current state in the patch area as a point zᵢ on one of the multiple Pareto frontiers to allow the selected solution to be changed (continuously or simultaneously) along multiple patches. To do this, the planner changes the position of a respective selector of a respective operating aid. A fuzziness interval is specified that is defined for a target criterion and also displayed accordingly in the interactive patch area; it covers at least two Pareto frontiers for at least two technologies. An equivalence of two technologies can be conveyed interactively to the planner, something the planner would not be able to recognize without this GUI.

A new use of a mouse pointer is included here from claim 25, without reference signs.

Embodiment examples of the invention are explained in more detail with the aid of the Figures. All explanations are equally applicable to the disclosure, but they are not to be interpreted in such a way that they must be included as necessary elements of the claims. All of the following examples remain examples even if they are not explicitly preceded by “for example.”

FIG. 1 is a schematic view of a sample sequence of a process of one or more embodiments of the invention. Here with patches as technologies on a display device with display 10 (represented by the four dashed corners). An initial system state is shown here.

FIG. 1A helps with understanding the Pareto frontier—in the example of Pareto frontier 201. A large number of grid points 201₁ to 201₁₀ can be seen. Therefore, the frontier is by no means continuous but instead is only functionally connected, which is why it is also shown as a (thin) line in FIG. 1, which is not really a line. The Pareto frontier can also be thought of as a projection on the n-dimensional space in which it exists, whereas it can only be visualized as a projection.

FIG. 1B illustrates reachable points on the multidimensional Pareto frontiers in two partial images of FIG. 1b1 and FIG. 1b2. The current position (the navigated point) is in the crosshairs in FIG. 1b1. The slim, upright white triangle indicates the points that can be reached. The larger hatched zone around marks the unreachable points, more of a projected area. Color coding not shown here can be used to represent a dimension that is not visible here yet still present. Uncertainties are represented in five “dots,” shown as small squares (cubes). The center of each of these is the expected value; four arms in 90° orientations each indicate areas of uncertainty as an interval. FIG. 1b2 shows the settings for FIG. 1b1; the sliders there can be configured in two ways: according to the target (objective) or the input (input I1, I2 and I3). There should be at least two of them.

FIGS. 1C1, 1C2 show the Pareto frontiers with their grid points, which can be interpolated between them. The shapes of Pareto frontiers 101 to 501 can be seen from FIG. 1. They are referred to as patches in the legend. The current point, shown with an “X,” is mapped by the sliders in FIG. 1C2.

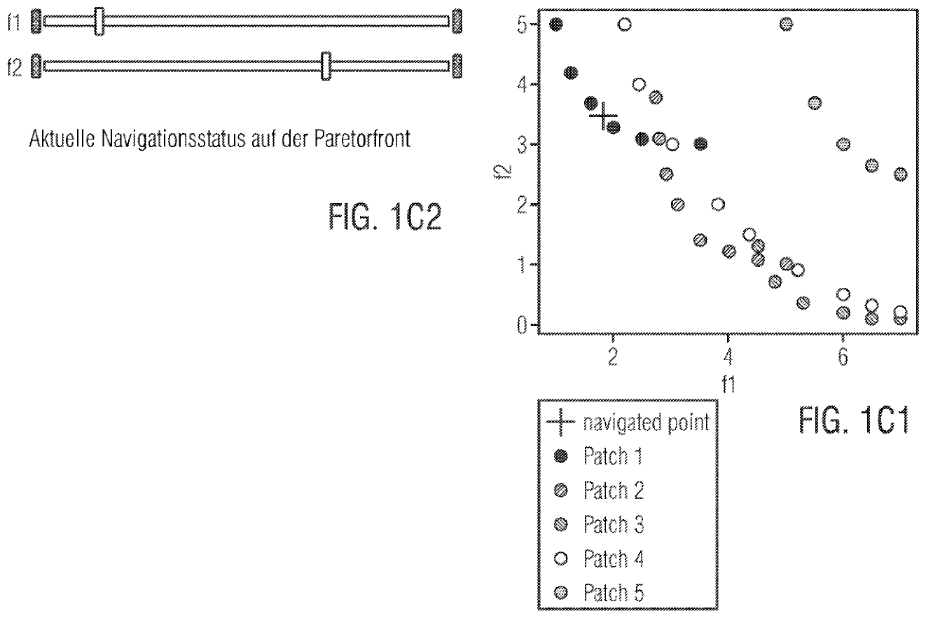

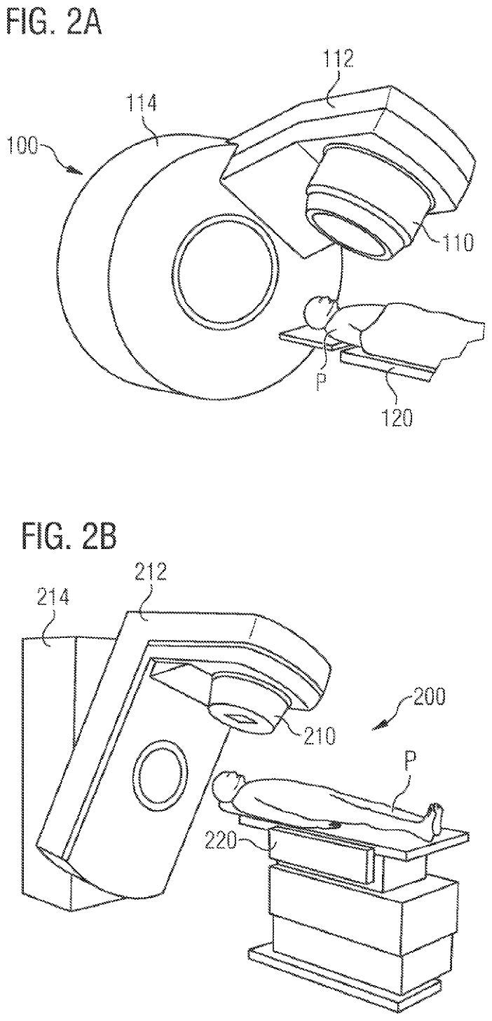

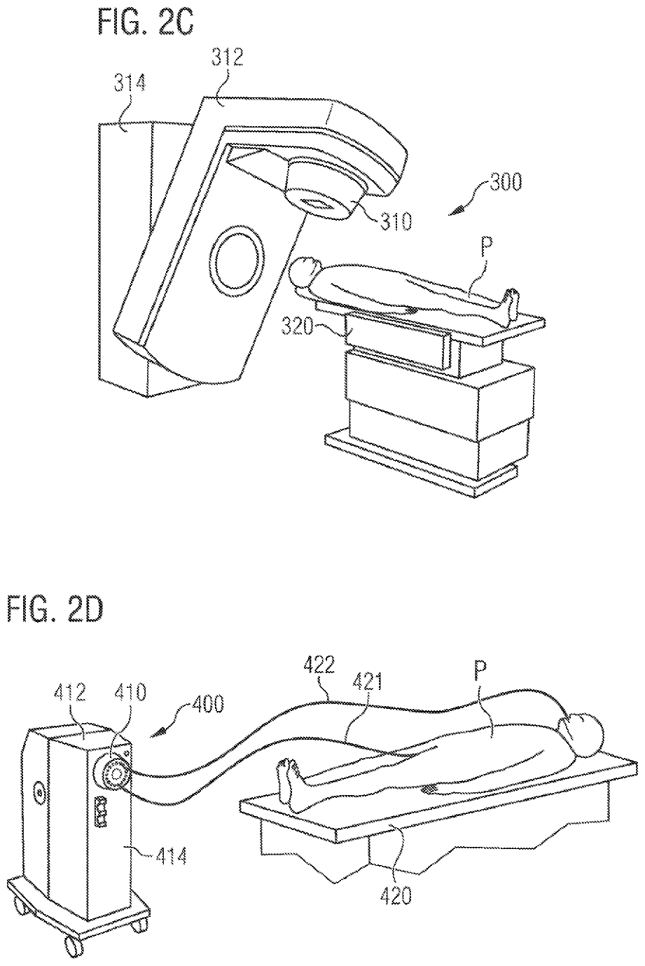

FIGS. 2A to 2D are different radiation devices 100, 200, 300, and 400, which provide different radiation devices.

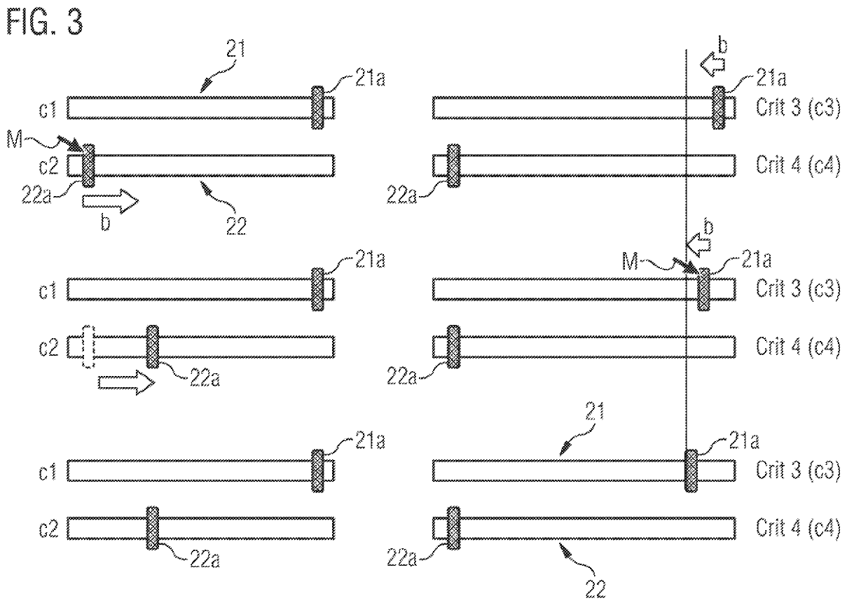

FIG. 3 illustrates a diagram of the movement of operating aids 21 and 22 in several image sections.

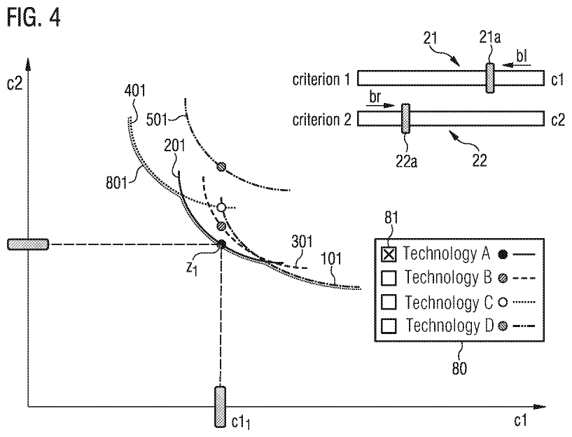

FIG. 4 illustrates an advanced system state on display device with display 10, based on the system state in FIG. 1. In addition, an optional resulting Pareto frontier 801 (as resulting patch 801) is shown here (in the form of a closely dotted line).

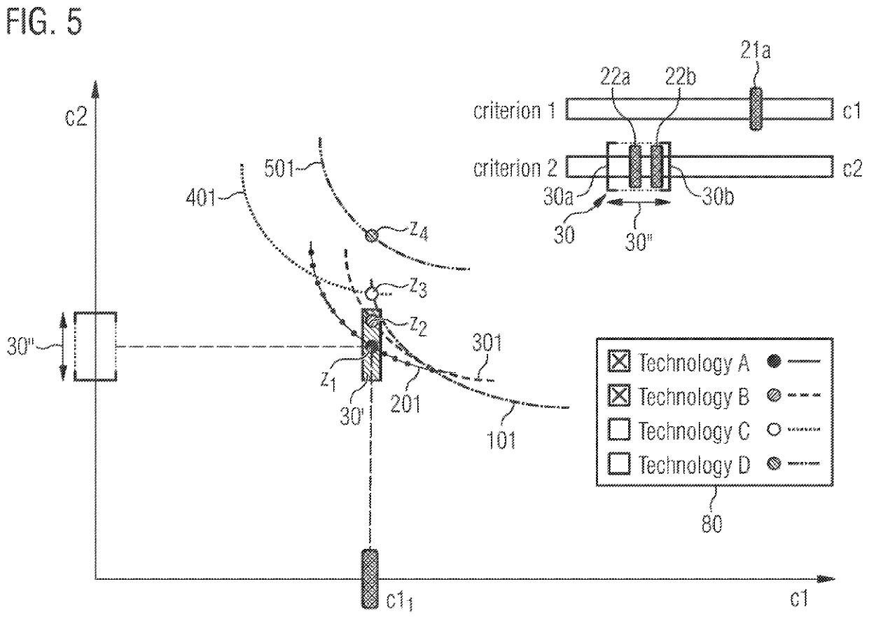

FIG. 5 illustrates another system state in which a similarity area 30′ has been opened at value c1₁. The similarity area is circumscribed here by a (vertical) strip 30′, and its length 30″ corresponds on the second axis as second criterion c2 to the section between ends 30a and 30b.

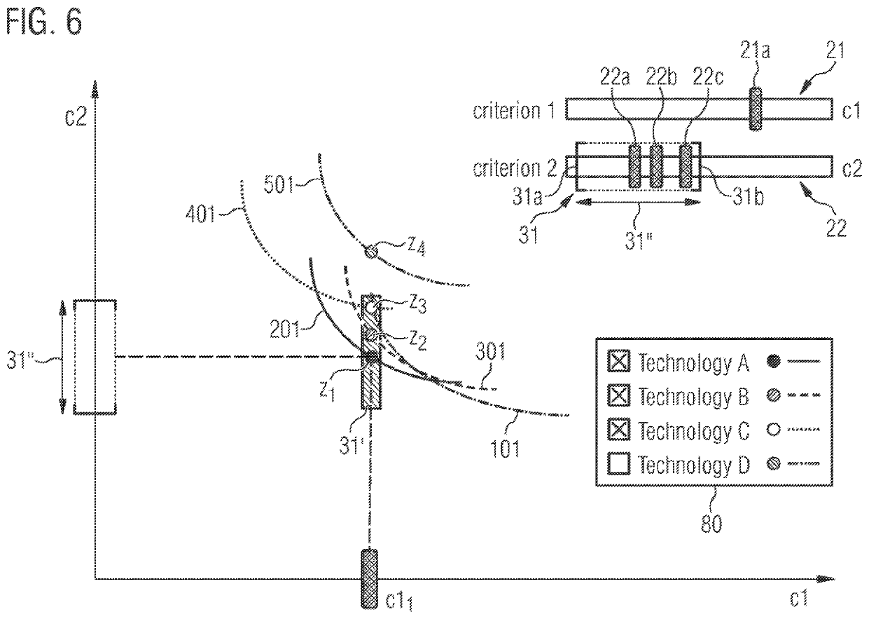

FIG. 6 is yet another system state in which the similarity area has been extended to a longer field 31′, corresponding to the increased length 31″.

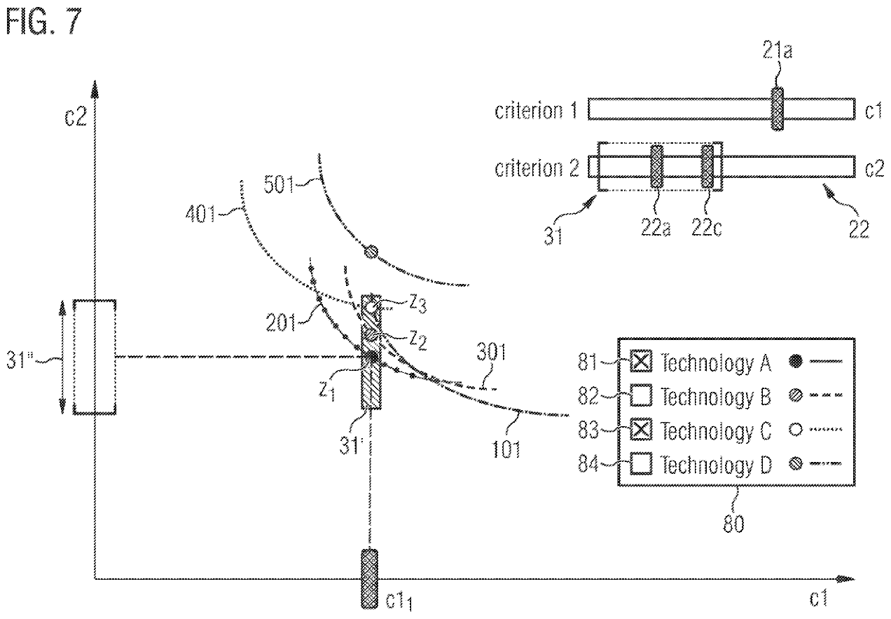

FIG. 7 illustrates the system state of FIG. 6, whereby one of the technologies has been deselected via a visual, interactive field 80 as a function field, so that this technology no longer appears or is no longer displayed in operating area 2.

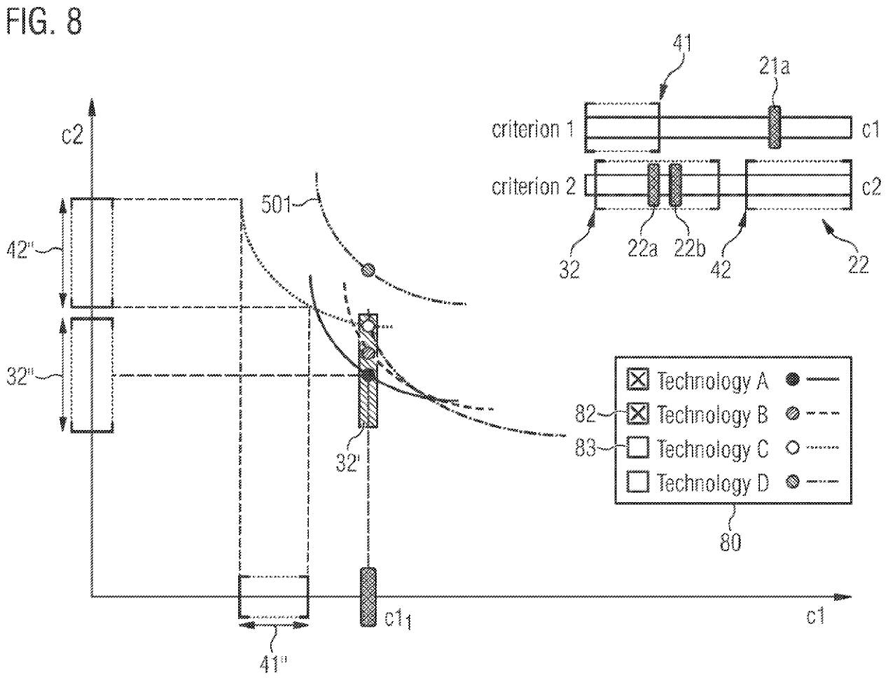

FIG. 8 illustrates the system state of FIGS. 6 and 7 and is an entire patch, patch 501 here, with the exception of the display in operating area 2, which leads to the restriction of selection options labelled with the areas 42″ and 41″.

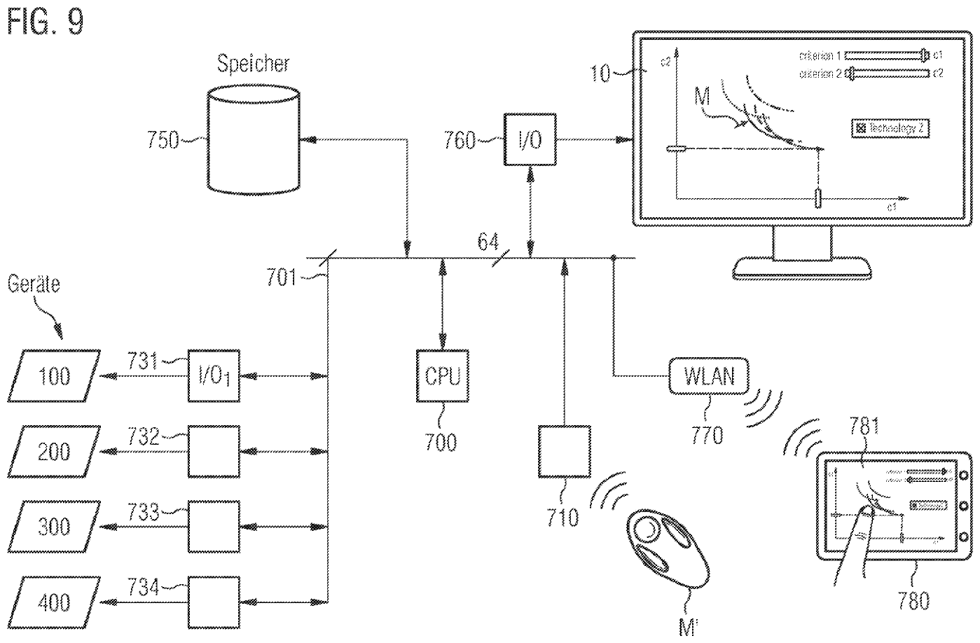

FIG. 9 illustrates a technical-functional data system with a computer, memory system, and input/output device with a display 10. Display 10 can be a normal display device or a touchscreen giving navigating control over the system state depicted in FIG. 1 with a finger (as a pointing device) instead of a mouse pointer. Here, a “pointer” is to be understood as a “function pointer,” such as a mouse pointer, a finger, or a laser pointer, each of which combines the pointing function with the operating function (at the location of the pointer on the display).

FIG. 1 illustrates the input/output device, which has a display 10. This display 10 is symbolized by the four corners indicated. It also results from the system structure of FIG. 9 with which the functions described below are achieved (or are achievable).

FIG. 1, enlarged, shows an operating area 2 at the top right and, taking up more space than operating area 2, a patch area 1 on the left. Operating area 2 is outside patch area 1, which means that these two do not overlap in a functionally obstructive manner. They appear visibly separated from each other for the planner, whereby their position is only selected in the example in FIG. 1 in such a way that the patch area is arranged at the bottom left and the operating area at the top right (on the visible surface of display 10). There is a visible and visually delimited field 80, which identifies technology Z as a functional field. This technology field, which in general is also called function field 80, is explained and expanded upon later.

Operating area 2 and patch area 1 are functionally coupled with one another. This functional coupling bears explaining. Multiple Pareto frontiers are shown in patch area 1; in the example, these are Pareto frontiers 101, 201, . . . to 501.

Also shown in the patch area are two axes c1 and c2, which are perpendicular to each other and represent two criteria c1 and c2. Criterion 1 (also c1) and criterion 2 (also c2) are shown, which are visible and navigable in operating area 2.

An intersection can be found at the currently selected point zᵢ. This selected point results from the settings of both operating aids 21 and 22, which can be seen in operating area 2. Criterion 1 is also called c1 and is plotted horizontally in patch area 1. Criterion 2, also known as c2, is plotted vertically in patch area 1.

In operating area 2, two settings are assumed for the two criteria and with the two operating aids 21 and 22; these settings can be selected using operating elements 21a and 22a and are also shown selected here. This setting results in the selected point zᵢ of technology Z, which technology Z is symbolized by 101 as a Pareto frontier, when operating area 2 is functionally coupled with patch area 1.

The concept of the Pareto frontier also bears explaining. The Pareto frontier is a representation of all technical variables that represent a therapy and, by definition, cannot be improved by multiple criteria. Such a multi-criteria optimum is characterized by the fact that there are no better solutions than those that lie on the Pareto frontier. No value (criterion) can be improved without worsening another criterion.

The concept of a solution is to be understood in such a way that a technically defined therapy is represented as a fractional treatment (as fractional “sessions”) over multiple days by a point on the Pareto frontier. This is therefore a treatment divided into time-spaced fractions. In the example, it can be at least 30 days, which is fractionated with a daily use of less than one hour—it is usually significantly less.

Between 30 and 40 of these daily fractions can take place in succession, usually not on weekends or at fixed times on each day of the week. This means that a total dose or total radiation exposure of between 60 gy and 80 gy can be reduced to a maximum of 2 gy per day.

The specialist speaks of fractionating in daily doses and can thus enable the patient to receive the lowest possible radiation exposure with the highest possible effect on the target, which is to be reduced or, if possible, completely eliminated by the radiation.

The planning of such a course of treatment is carried out by means of the “solution,” which defines this course of treatment for the specific patient, represented here by a single point zᵢ on one of the Pareto frontiers. This point is therefore not representative of just one patient but of a whole series or sequence of system settings on the technical devices that will be outlined below.

This representation is a highly abstracted form of the conveying of a solution that is precalculated and stored in a memory 750 of the bus system shown in FIG. 9.

It can be seen that each additional point on the Pareto frontiers also represents a solution, so that the concatenation of all solutions on a Pareto frontier defines the curve of this Pareto frontier. If the planner (as the user of the tool for designing and fashioning a therapy as a treatment plan according to FIG. 1) changes the patch, they also change the device or the regime on the same device with which the radiation is generated for the patient and administered in the course of the therapy.

It bears emphasizing again here that it is not the treatment as such that is patented, but rather the planning of this therapy. What cannot be avoided, however, is the reference to the subsequent effect of the planning on and with the patient, who is symbolized by P in the following pictures.

We now return to the functional coupling of patch area 1 with operating area 2, with a view to FIG. 1. Setting the two operating elements 21a and 22a defines point zᵢ in the patch area. The settings of operating elements 21a and 22a are still represented on the two axes of criteria c1 and c2; the selected x-y representation clearly indicates point zᵢ on patch 101, with the result that this plan with its technology Z is currently selected.

Multiple technologies are shown, which are symbolized with A, B, . . . Z. These technologies may include the technologies described at the beginning of the application, whereby each technology may also include a regimen. Technology is therefore to be understood both as the choice of the type of radiation therapy (radiotherapy) as well as the therapy regimen within the same type of radiation.

Numbers 1 to 6 at the beginning of the application illustrate protons and photons as radiation in nuclear medicine, each accelerated by a linear accelerator, whereby there are other radiation generators and other types or kinds of radiation in nuclear medicine as well. In addition, there are accelerated electrons, accelerated heavy ions, as well as the protons and photons mentioned in the examples. The radiation emitters can also be used in brachytherapy. These devices use advanced radiation particles (subject to radioactive decay) for the direct (internal) emission of gamma rays. The radiation bodies can have a cylindrical shape with a length of less than 5 mm and consist of cobalt-60, iodine-125, or iridium-192, preferably surrounded by a titanium shell.

Regimens are the ways in which a particular type of beam is used, such as with two rotations, for example with nine fields and nine fixed angles, or with nine fields and angles different than the nine fixed angles mentioned.

According to the previous description, operating area 2 includes two linearly movable sliders as operating aids 21, 22, whereby each slider is currently assigned an operating element 21a or 22a, which is to be referred to as a selector. Examples of operating aids are sliders; examples of operating elements are digitally displayed control buttons, also called selectors.

This selector can be used to improve a respective criterion in operating area 2 or to worsen it in the corresponding opposite direction. According to existing conventions, a movement to the left is an improvement, so that criterion c2 (on the associated c2 axis) is already fully optimized (as it is on the left edge of operating aid 22). For the example on the next page, the “selector of the slider is close to the left stop.”

In FIG. 1, the corresponding illustration is shown in patch area 1, where selector 22a has the lowest value. Increasing the value of criterion c2 in the planning leads (later, not actively included here) to a deterioration of the therapy, but since criterion c1 must also be observed and can still be improved on the basis of the position of slider 21 and its selector 21a, a movement is to be expected, which is explained with the aid of the following Figures.

In an overview, operating element 21a moves to the left (also to the left on axis c1 in patch area 1). Operating element 22a moves to the right (upwards on axis c2 in patch area 1). The orientation of operating aids 21 and 22 in operating area 2 can also be swivelled by 90° so that left/right becomes down/up. This can also apply to each of the operating elements 21 and 22, which are aligned in such a way that they appear like a Cartesian coordinate system.

This movement is explained in individual steps in FIG. 3 in six image sections; more on this later.

Before this movement is explained, the devices associated with Pareto frontiers (patches) 101 to 501 of FIG. 1 bear explaining, whereby four devices are shown as an example, which are not intended to be exhaustive, but merely exemplary.

The first device 100 of FIG. 2A is a device 100 for emitting protons during or for radiation therapy administered to a patient P positioned on a table 120 (ready for treatment). A first support body 114 is rotatably mounted, and a radiation head 110 is arranged on it, which is rigidly connected to support body 114 via a bridge 112. The angle of radiation head 110 relative to patient P can be adjusted via the support body. The radiation dose and the distribution of the radiation within the proton beam from radiation head 110, which is not shown, can also be adjusted. All these values are represented in a Pareto frontier for a patient over the entire course of a fractional radiotherapy. In the example, this can correspond to Pareto frontier 101 of FIG. 1. The technical values of radiation head 110 are set via an I/O interface 731 from 64-bit bus 701, which can be seen in FIG. 9.

FIG. 2B shows another radiation device, here a photon radiation device 200. Patient P is placed on a base 220, and radiation head 210 for emitting the protons is arranged on an L-shaped bridge 212. Bridge 212 is rotationally connected to a stationary base 214, relative to which it can be pivoted. Here, too, the settings of the beam emission of radiation head 210 can be prescribed, and they can be prescribed for a fractionated treatment of a Pareto frontier, such as Pareto frontier 201 of FIG. 1. The technical values of radiation head 210 are set via an I/O interface 732 from 64-bit bus 701, which can be seen in FIG. 9.

FIG. 2C shows another radiation device 300. Here, too, patient P is laid out on table 320. Bridge 312 is arranged so that it can pivot on a base 314, and radiation head 310 is designed to emit accelerated photons. The emission of the photons from beam head 310 is adjusted so that they are matched to the target and the risks in such a way that the target is primarily covered by the beams in its volume during the fractionated treatment session and the risks are shielded from exposure to radiation. To this end, a multi-slat collimator can be provided, which is placed in radiation head 310 and whose slats are adjusted in such a way that they are brought into any desired shape in order to open up a suitable area as free space between them, corresponding to the target volume in the best possible way. The slats themselves are beam-shielding—made of tungsten, for example—so that the shape of the beam can be adjusted almost at will. In addition to this shaping adjustment of the radiation volume (the radiation area, actually), a regimen can be used to specify the fields and angles at which the irradiation occurs. The technical values of radiation head 310, such as the multi-slat collimator, are set via an I/O interface 733 from a 64-bit bus 701, which can be seen in FIG. 9.

Another radiation device can be seen in FIG. 2D. It is a radiation device 400 for emitting internal radiation, whereby the brachytherapy described at the beginning of the application is used and multiple tubes in the form of catheters 422 and 421 extend from a distributor head 410 (also called a “radiation head”) and are placed at a respective radiation site located in patient P. Patient P lies on a treatment table 420. Small radiation bodies, not shown here, are moved out of a radiation safe 412, which is delimited in actual device volume 414 in a radiation-safe manner, and brought along catheters 422 and 421 to the location where the radiation is to be applied as an internal source of radiation in the patient for a certain period of time. They are then retrieved and secured again in radiation safe 412 to block the parasitic emission of radiation. The technical values of radiation head (e.g., the dwell times in the body) are set via an I/O interface 734 from 64-bit bus 701, which can be seen in FIG. 9.

The beam emission of the radiation device of FIG. 2D can correspond in its planning to Pareto frontier 501 of FIG. 1, for example. The previously described radiation device 300 of FIG. 2C can plan its radiation output to correspond to Pareto frontier 301, for example.

Other devices, not shown separately here, can also be provided, and their beam emission can be the subject of Pareto frontiers, as presented by Pareto frontier 401 of FIG. 1.

FIG. 3 illustrates the change in operating elements 21a and 22a of the two criteria c1 and c2 in (assumed) slow motion or in small steps. Three image sections in the left half of FIG. 3 are representative of this. A function pointer M is assumed, which is explained in greater detail later.

Starting from a setting of operating elements 21a and 22a on operating aids 21 and 22, the initial state depicted in FIG. 1 is shown. Criterion c2 is set to the best possible value, as it is furthest to the left. In the example, worsening of this value is to be brought about by movement b, whereby operating element 22a is to be moved to the right by a mouse pointer M or, in the case of a touchscreen 780 according to FIG. 9, by activating and swiping with a finger. In the center display, it moves from its original location in the upper image section to a location further to the right. This can be shown using an optical fading technique; the previous location fades and the new location appears.

Movement b is completed or finished when operating element 22a has reached its new position, as shown in the third image section on the left. The original position, which was still indicated in the center image section, can no longer be seen. In one movement from the first to the third image section, there are many intermediate states that are not shown here.

In the example, it is assumed that criterion c1 does not change when operating aid 22a is changed in this way, which will not be the case in practice since a change in one criterion will also change the other criterion, as they are functionally connected to each other via Pareto frontier 101, i.e., they are functionally coupled with one another, although not physically connected.

The actual functional connection takes place through specification of the predefined solutions on the Pareto frontier, patch 101; if the planner changes a solution in one criterion, a second, third, and/or fourth criterion also changes, and thus also the second (or more) operating element(s) in precisely this second criterion or for precisely this second criterion c1.

FIG. 3 shows two other criteria c3 and c4 in the right half. Here you can see a movement of criterion c3 from right to left, i.e., toward a better value. Here, too, criterion c4 is shown unchanged with operating element 22a for illustration purposes, represented by the unchanged position of operating element 22a in slider 22.

Movement b is shown running slowly and gradually up to the marked vertical auxiliary line and the new position of operating elements 21a on operating aid 21 (also as selector and slider, which are examples of them). Under the planner's control, function pointer M takes operating element 21a of operating aid 21 and sets it to the position of the third image section.

Taking into account the options that multiple criteria can be changed via operating aids, although only two criteria c1 and c2 are explained in detail in the examples selected here, it can also be understood with the aid of FIG. 3 that two further criteria c3 and c4 can be changed—or that criteria c1 and c4 are shown in operating area 2, functionally coupled with the representations of Pareto frontiers 101 to 501 (in axes c1 and c4) that are then fitting in patch area 1.

Typically, five to six criteria are compared. The Pareto calculations for specifying the predefined solutions use between ten and fifteen criteria. There are three to five criteria in mutual competition, between which a compromise must be found in order to find and define a solution with which the planner is so satisfied that they submit this plan to the doctor for review and subsequent approval.

Functionally, the criteria are to protect the risks in the immediate vicinity and to damage the tumor as much as possible in the case of cancer therapy, i.e., to destroy it as best as possible using radiation. The areas farther away from the tumor are less critical. There are often three decision criteria with regard to what the rectum, prostate, and bladder receive as a radiation dose and whether a given plan is good for a patient.

It was described that one of the criteria can be the treatment time. The treatment time plays a role for the patient registered for a therapy, and it played a role in how good this therapy is for the patient, for example how much the time that a patient has to wait before receiving the best possible therapy for them can be reduced. Here, the aim is for the quality of the therapy to also include the fact that the patient experiences the shortest possible waiting time when waiting for the therapy developed for them (time to treatment). This goes hand in hand with ensuring that the available equipment (radiation equipment) is used and deployed in such a way that the available equipment potential can be utilized for the patient and that preference is not given to certain equipment on which or with which multiple patients are to be treated. This will lead to bottlenecks if other devices cannot or could not carry out the fractionated time elements of a therapy in the same or at least a very similar way.

To achieve this, FIG. 5 shows a fuzziness interval 30 of length 30″ (shown here as a vertical strip 30′ in patch area 1), which is introduced for a criterion, in this case c2. For better understand of this fuzziness, FIG. 4 first merits an explanation, in which no such fuzziness interval 30 is introduced, but instead a resulting Pareto frontier 801 is introduced as an additional option.

The resulting Pareto frontier 801 uses sections of other Pareto frontiers shown (patches 101, 201 and 401) for three technologies shown, or it runs along these sections. It is a graphical option; the sum of the patches themselves—101, 201, and 401 here—also fulfills this task, as the intent is to functionally permit navigation to go beyond the limits of a technology.

In the example of FIG. 4, resulting Pareto frontier 801 shown contains sections of Pareto frontiers 401, 201, and 101. Resulting Pareto frontier 801 is the set of Pareto optima (Pareto set) of the technologies. Resulting Pareto frontier 801 therefore represents the Pareto optima of the considered Pareto optima of the technologies.

Resulting Pareto frontier 801 can also be added to the following FIGS. 5 to 8, where it has not been additionally drawn. For better clarity, it is slightly offset graphically from the Pareto frontiers of the technologies in FIG. 4. This can also be recommended in a representation on a display device 10.

In FIG. 4, the planner has set an improved value for criterion c1 by moving operating element 21a to the left. Due to the functional coupling and the curve of the Pareto frontiers, there is a shift to the right for criterion c2, whereby operating element 22a is automatically shifted to the right via the functional coupling, while operating element 21a is shifted to the left by the planner by clicking with the mouse or by touching and swiping/sliding. The two arrows br and bl show this movement. The movement was illustrated in FIG. 3, where the functional coupling was eliminated and only one operating element moved at one criterion.

FIG. 4 shows that, by moving (changing criterion c1 to an improved value), point z₁ controlled in patch area 1 is a different one. By moving operating element 21a to the left, point z₁ on Pareto frontier 201 moves from right (further down) to left (further up). In the example, this is represented at the point where the two operating elements 21a and 22a come to rest on the axes of criteria c1 and c2 in operating area 2.

What can be better understood with the aid of FIG. 1 in FIG. 4 is the movement of the generally positioned point zᵢ toward the point z₁ shown here. The patch has been changed from Pareto frontier 101 (patch 101) to Pareto frontier 201 (patch 201).

FIG. 4 then symbolizes as well that, at point c1, which is held by operating element 21a in operating area, there are multiple points that lie on other patches, such as the points on patches 301 and 101 (or 401). However, since these patches are not selected (see function field 80), technology A remains, which is assigned to patch 201 (and defines the two criteria c1 and c2).

This defines function field 80, which represents a number of technologies that can be selected and deselected by the planner. This selection is symbolized by an x in checkbox 81 (see FIG. 7). The assignment of the z points is also symbolized by the appropriate coloring (hatch marks) and the associated paths on which they can move, whereby each path is a Pareto frontier. Each path has its own line shape, also shown in function field 80, and this includes its own hatching of the point on the path. Point z₁ is filled in and on the continuous path, which corresponds to patch 201 and technology A. It is selected and can be navigated by the planner.

The technologies to be displayed can be specified in function field 80 as a visual field.

However, the selection of further technologies does not mean that multiple patches can be selected in FIG. 4, nor that multiple technologies are therefore available for a setting or can be selected for navigation. This requires the initially indicated function of extending a coordinate on a single target variable (a target criterion) to an interval on at least one of the axes, in this case criterion c1 or c2.

This setting is done in FIG. 5. The setting shows the same state as that of FIG. 4 with the three possible target points z₁, z₂, or z₃. Two of these three possible technologies are selected in function field 80 so that two technologies lie in the similarity area of a fuzziness interval 30, which extends the single coordinate of criterion c2 to a coordinate area. The extended range of length 30″ is the fuzziness interval (also fuzziness range) with a lower border 30a and an upper border 30b. Since the convention requires an improvement to the left, the terms upper and lower range are misleading, so they should be called left end 30a and right end 30b instead. Fuzziness range 30 extends between these two with a length of 30″.

The fuzziness interval on criterion c2 is also shown graphically in patch area 1, on criterion axis c2.

Length 30″ corresponds to the interval between left end 30a and right end 30b in operating area 2. This area is also shown via vertically extending field 30′ (shown with hatch marks), which in the example shown covers (or spans) two technologies: technology A and technology B. Selecting these two technologies in function field 80 gives the planner an additional option. Instead of just technology A along Pareto frontier 201, technology B, which runs along Pareto frontier 301, can also be selected, as it is very similar to technology A and Pareto frontier 201.

The left and right borders of the extended identity, now the similarity, can be made via a separate setting, which is not illustrated. This can be an additional operating aid that defines a threshold that can, and preferably should, be below 10%. Alternatively, it can be defined by clicking the mouse or by double-tapping and swiping on a tablet, i.e., with a touch-sensitive output/input device 780 as input/output device 10.

The term “function pointer” is used across all devices, combining the pointer and the function option. With an icon, the mouse indicates that holding down a click on the display allows a “Drag” function, while a double-click allows a selection. Similarly, the single tap or double tap of a finger works as a locator or activator on a screen-enabled device (e.g., a tablet). The same applies to the laser pointer as a pointing device, which is coupled with a function by on/off or the drawing of an symbol at the pointing location, e.g., as a small circle with a 360° movement in a clockwise or anti-clockwise direction.

Also shown in FIG. 5 is the appearance of the second patch frontier 301 and the further point z₂ covered by the fuzziness by showing a second operating element 22b. Both operating elements 22a and 22b lie within the two borders 30a, 30b of the extended identity or of similarity area 30″.

It has already been mentioned that field 30′ extended to length 30″ as a fuzziness interval 30 in patch area 1 leads to narrow field 30′, shown with hatch marks here, which covers at least two Pareto frontiers. Since the planner also controls the range or the size of the fuzziness, the user can achieve 30 by narrowing and expanding this fuzziness interval and can also see interactively whether there is any leeway in the planning process. If the fuzziness is too low, no further operating element appears in its control panel. If the fuzziness is greater, as in the example shown with the two ends 30a and 30b, at least one additional patch is available for selection, and thus one additional technology, as can also be seen in function field 80.

The technology corresponding to Pareto frontier 401 or Pareto frontier 101 cannot be achieved, as associated target point z₃ lies outside the expected identity, i.e., outside fuzziness interval 30′.

The fuzziness interval can be specified by a further slider, not shown in FIG. 9, or by an optically operable rotary adjuster that is functionally shown on the display. The planner can also select a discrete value range of, for example, 5%, 6%, 7%, 8%, and 10% and even 15% via a field of checkboxes. Clicking on a different percentage range than the one currently set deletes the setting of the previous fuzziness interval.

After specifying the clinical objectives as described and defining a list of possible technologies, the Pareto frontiers shown in the Figures are calculated automatically. The planner is offered all technologies for simultaneous navigation and, by means of interactive operability, has the option of leaving the identity area in favor of a similarity area, called fuzziness, in order to have multiple technologies to choose from at the same time. To this end, it is also evident that the planner can switch from one patch to the next, i.e., from one Pareto frontier to another Pareto frontier, and is therefore not restricted to one technology and one Pareto frontier. This makes it possible to have a non-dominant set of the union of all patches available for navigation.

In a practical sense, FIG. 5 would mean for a planner that it is not only possible for a patient P to be treated with the technology corresponding to Pareto frontier 201 and therapy z₁, but also served (treated) with therapy z₂ on another device or on the same device with another treatment regimen in fuzziness interval 30.

Overall, it is not just the individual patient that is relevant for a hospital but the sum of all patients who can be treated with fractionated therapies over a longer period of time, for example 30 to 40 days. With regard to the hospital, this is not limited to the 30 to 40 days either, but can be planned over the entire year, and each of the plurality of patients treated is scheduled in the hospital's entire equipment pool (of available radiation equipment) via the fuzziness interval in such a way that not only the best and most optimal device, which is usually the most technically advanced device currently acquired, is available and used, but also that many other devices are available for the patients or the specific patient over the described fuzziness interval, which improves the overall quality of their therapy, as the waiting time until the start of their treatment is reduced.

Function field 80 can also have the function of a preview, as shown in FIG. 5, in which four technologies A to D are offered in function field 80.

These multiple, here four, technologies are the ones that are possible via value c1₁ because they are all reached by this value, i.e., target points z₁ to z₄. All these technologies appear automatically controlled by the associated software in extended function field 80, compared to technology E from FIG. 1. Point zᵢ there and the position of operating element 21a do not permit any additional intersections, so no additional Pareto frontiers can be selected, unlike in FIG. 5, in which the position of operating element 21a (with criterion c1) offers four possibilities, albeit with a different fuzziness interval 30.

FIG. 6 illustrates the system state of FIG. 5, and an extended field 31′ is specified, which is created by moving two borders 31a and 31b in slider 22. The span of this extended similarity area 31″, as also shown on axis c2 of second criterion c2, is larger than span 30″ in FIG. 5.

More Pareto frontiers are achieved, and there are more intersections z₁, z₂, and z₃. These points, target points, or intersections are also displayed interactively and visually on the slider or in the slider for criterion c2. Two additional operating elements 22b and 22c are created there with existing operating element 22a, which represents intersection point z₁.

The extended similarity area extends the first similarity area of FIG. 5. FIG. 5 itself was already a first similarity area that went further than the identity of the depiction in FIG. 4 in order to cover multiple technologies. All these extensions, which go beyond a single technology, are conceptualized by the fuzziness as an interval, which continues to be controllable in itself, as described below.

Fuzziness interval 31 with extension 31″ due to vertical field 31′ at target value c1₁ of criterion axis c1 can also be changed, as already explained above; it can be made larger or smaller, which is something the planner can do. This change can be made by clicking and dragging borders 31a or 31b, individually or together. It can be done on a touchscreen 780 by activating a border with an initial touch (tap) and then moving it with a swipe.

The borders, one or both, can also be changed by a separate slider, linear or curved. This slider is not presented separately but is left up to the technical understanding of the reader.

What all variants have in common is that length 31″ is changed.

In FIG. 6, it has already been enlarged compared to FIG. 5. By zooming in, more technologies appear in function field 80, which is part of the overall display on display/screen 10 as a “functionally coupled” function field. It can therefore influence the ability to interact with the planner.

Function field 80 can display further technologies after similarity area 31 is expanded. In the example, accessible technology D is also included in the function field under technologies A, B and C.

This intersection is named z₄ in patch area 1 and is located on Pareto frontier 501. It cannot currently be reached by second operating aid 22; only three operating elements 22a, 22b and 22c are specified as the respective selector here. Nevertheless, function field 80 indicates in advance that a further technology is available that cannot be achieved due to the limited length of vertical elongated field 31′.

In this respect, function field 80 is therefore an indication of what could still be, what is brought to the planner's attention interactively, but which cannot currently come to fruition due to the interactions in or with the control panel and here in particular due to the (limited) extension of fuzziness interval 31.

Hypothetically, it can be assumed that a further shift of border 31b would also lead to patch 501 being reached. However, this could overstrain the similarity. Preferably, the selected area of similarity is below 10% points. It is therefore very limited in order to maintain the similarity in terms of functional significance. It may be recalled that a similarity used interactively here to find alternatives to treatment plans is technically conditioned, i.e., a technically meaningful similarity is sought that would open up ways out of existing constraints. A normal planner would not be able to guess these ways out just from the known equipment available at the therapy location (usually the hospital). It is also unclear to the planner what technical similarities exist or could exist between a proton device, a neutron device, and/or a photon device without using the illustration in FIG. 6, for example, which provides the associated Pareto frontiers 101 to 501 for interactive navigation.

It has previously been explained how similarity area 31 is enlarged as a fuzziness (interval) based on FIG. 6. In this enlarged similarity area, however, further options are available to the planner, such as the option that a certain technology, which is actually shown as being available in the similarity, is not to be selected.

This is explained below in FIG. 7.

FIG. 7 shows the system state from FIG. 6 and explains how the representation in FIG. 6 is different. The same similarity area 31 remains, which also has a vertical extension 31″ as shown in FIG. 7. In this similarity area with the aforementioned extension, two operating elements 22a and 22c are located in operating area 2 on operating element c2. Therefore, no three elements 22a, 22b, and 22c from FIG. 6 are shown here, but operating element 22b has been omitted because the planner has deactivated or “clicked away” technology B in interaction field 82 in function field 80.

Checkbox 82 for technology B, representative of Pareto frontier 301 and intersection z₂ is not available to the planner. The planner can select the technologies of available intersection points z₁ and z₃ from the position of operating elements 22a and 22c. For the technology of intersection z₃, the planner could preferably use Pareto frontier 401 and select the associated device, as this Pareto frontier has a lower gradient at intersection z₃, which can be a criterion for selecting the associated Pareto frontier.

It is also clear from this explanation that all elements of the image display are functionally coupled with one another.

Selecting or deselecting a technology in function field 80 affects the number and presence of operating elements in operating area 2. Changing similarity area 31 has an effect on the vertical extension of fuzziness interval 31′ seen in patch area 1 and thus on the number of possible and displayed operating elements 22a, 22b, and 22c, which in turn are shown as selected or deselected or not displayed, depending on the labeling in the checkbox of function field 80.

The checkboxes are to be labelled 81 to 84 as interaction fields; checkboxes 81 and 83 are selected in FIG. 7, corresponding to technologies A and C and consequently to operating elements 22a and 22c of operating aid 22 for criterion c2.

Even if only two criteria c1 and c2 are represented in the Figures, more and other criteria can be depicted, as explained in FIG. 3.

The functional link between operating area 2 and patch area 1 as well as function field 80 has been previously explained and is corroborated here. It is not only possible to operate via operating elements 22a or 22c, with which a movement can take place via the functional link on a respective patch (i.e., a respective Pareto frontier)—in this case with regard to the selected point of, say, z₂ when operating element 22c is moved. This point does not move horizontally but, in conformity with all other criteria, in conformity with criterion c1 here, i.e., on Pareto frontier 301. The Pareto frontier is also visible in all other criteria but is not shown here as well due to the two-dimensionality, although it is easy to conceive of (for an expert).

Operating or changing the position of operating element 22c can also be done by changing the position of the z point itself with a mouse pointer or, in the case of a touch panel, by active use of a swiping or sliding movement. The functional coupling ensures that the position of associated operating element 22c on the operating aid for criterion c2 is also changed.

This change affects target point z₃ since it is selected via technology C and checkbox 83, i.e., it becomes and is visible as operating element 22c in operating aid 22. A displacement of the other visible point z₂ is not displayed in the operating area on operating aid 22, as its technology (that of Pareto frontier 301) is not selected in function field 80. Corresponding checkbox 82 is deselected. This shows that operating area 2, which is primarily intended for planner operation, is only an option. It can also be operated from patch area 1, with a view of interactively available target points zᵢ shown.

FIG. 8 shows that excluding a patch can lead to entire areas being blocked.

These locked fields are shown with 41 and 42 on the two operating elements 21 and 22. They are depicted from operating area 2 in patch area 1 by extension 42″ on criterion axis c2 and extension 41″ on criterion axis c1.

These two exclusion areas follow the deselection of technology C via checkbox 83 in function field 80. As taking this technology into account for planner interaction is not intended at this time, corresponding sections 42″ and 41″ are not available on criteria axes c1 and c2. The selection options are thus limited, but it is still possible to select fuzziness interval 32 for criterion c2, with value c1₁ remaining constant on the axis of criterion c1.

This field 32′ corresponds to field 31′ in FIG. 7, except that technology B is selected here via checkbox 82 so that another operating element 22b also appears in operating area 2 on operating aid 22, whereas operating element 22c is missing due to the deselection of technology C by deactivating checkbox 83.

In the example shown, the exclusion of patch 401 leads to a restriction of the selection options. Excluding this patch eliminates the “best options” of criterion c1.

Associated fields 42″ and 41″ can be marked in color on the display screen, ideally in a signal color to indicate their unavailability in the patch area, but also with a signal color in associated exclusion intervals 41 and 42 in operating area 2.

If the other criterion c2 is fixed on its axis in all examples, the vertical similarity fields described above extend in a horizontal direction. These orientations are disclosed accordingly as well. The description of a direction or orientation does not entail a limitation of this.

FIG. 9 shows a network-orientated depiction of the control system via a bus 701 and the digital components used that are coupled to it. In the preferred example, bus 701 is a 64-bit bus via which the components communicate with one another. It is bidirectional. For the computing capacity and computing function, CPU 700 is specified, which reads functions 101, 201, etc. displayed on display 10 from memory 750 and forwards them to display device 1/O 760 via bus 701. Depending on the technical type of display 10, the data is processed and depicted in display device 760. The navigation environment of FIG. 1 is shown. It is operated via function pointer M and moved and activated by the planner via a mouse device, shown here in the example as trackball M′ (selection of a function at a location of pointer M). Trackball M′ is coupled by radio to a receiver 710, which converts its movement signals and forwards them to bus 701. This coupling is not bidirectional.

Mouse device M′ is assigned to display 10, e.g., as the trackball, which enables the functions of pointing and operating (triggering a function) at the location where mouse pointer M is displayed.

Mouse pointer M is also used to operate both operating aids 21 and 22 for criteria c1 and c2.

An alternative display 781 is that of a tablet 780, which is given the same representation of FIG. 1, which is shown on touch-sensitive display 781. As explained above, the screen display or tablet display has a patch area 1 and operating area 2. Tablet 780 is coupled with bus 701 via a Wi-Fi coupling (or connection) with a Wi-Fi transmitter and receiver 770. The mouse pointer is replaced by a finger (as shown) and controlled by gestures (e.g., swipe, tap, double tap) on display 781 of screen-enabled or touch-sensitive tablet 780.

Technological radiation devices 100 to 400′ are each coupled to bus 701 via one input/output device 731, 732, 733, and 734, respectively. Each of these input/output devices is bidirectional, meaning that it can preset parameters from bus 701 to the respective device for its use after settings are made; preferably, each of these devices 100, 200 has sufficient memory of at least 500 GB so that it can store the preset parameters of the subsequent therapy and set them independently, i.e., autonomously, during the therapy, especially for fractionated patient sessions over the planning period. In the example, I/O₁ sends the setting parameters to device 100 for the emission of protons. This is then able to apply the parameters of the proton device 100 that are to be set—which are actually already prescribed—during the (fractionated) therapy of patient P spread over days. In other words, they are the doses and intensities and directions of proton radiation (in radiotherapy) to which this individual patient P is to be exposed.

This example can also be controlled in such a way that the parameters are stored in memory 750 for the duration of the fractionated therapy and are only to be transmitted to radiation device 100 at the times before the respective therapy session. This fractionated therapy is fractionated programming of respective device 100 to 400 (akin to the fractionated data transmission of each currently required time section of the therapy, such as 30 days). The actual therapy is performed automatically by device 100, without the previously completed planning.

In the same way, the other devices 200, 300, and 400 are also programmed, prepared for therapy, and conditioned with regard to data technology.

It bears emphasizing that no therapy is performed during the planning stage; the therapy is already fully planned and functionally prepared (i.e., fully planned) before the respective device performs this radiotherapy on the patient. The latter therapy is not claimed.

Navigation via the patches shown on display 10 can only be completed by the planner in order to transfer them later to the associated devices via the respective input/output devices.

The calculation of CPU 700 results in a graphical definition of a similarity area 31, which was previously described as a fuzziness interval. It is not shown on either display 10 or touch-sensitive display 781 of tablet 780.

1. Procedure for designing or fashioning a therapy as a treatment plan, before treatment, and with interactive navigation on a display device (10)
(a) whereby multiple technologies (A, B, . . . Z) at least one radiation device (100, 200, 300) are available for selection, including at least one technology (A) with a radiation device (100) for emitting protons and/or at least one technology (B) with a radiation device (200) for emitting photons;
(b) after the designing or fashioning, for a person (P) who is to be treated with therapy that can be provided by the radiation device (100, 200, 300), whereby the designed or fashioned plan defines a plurality of technical settings that are adjusted on the radiation device(s) of the selected technology/technologies;
(c1) for at least some of the technologies (A, B, . . . Z), one Pareto frontier (101, 201, 301) each is shown on the display device (10), each of which is referred to as a patch;
(c2) an operating area outside a patch area on the display device (10) shows a number of operating aids (21, 22, . . . ), especially as sliders, each of which represents a criterion (c1, c2, . . . ) that is improved when a selector (21a, 22a, . . . ) of the respective operating aid is moved or operated in one direction or worsened when it is moved or operated in the opposite direction;
(c3) and, for interactive communication with a planner, a respective selector (21a, 22a) highlights a solution selected in the current state in the patch area (1) as a point (zᵢ) on one of the multiple Pareto frontiers (101, 201, 301), whereby the selected solution is changed along multiple patches that navigably depict the multiple technologies (A, B, . . . Z) when the planner changes the position of the respective selector of the respective operating aid;
(d) with a fuzziness interval (30, 31, 32) that is defined for a target criterion (c1, c2, . . . ) and that is also displayed accordingly in the interactive patch area and covers at least two Pareto frontiers (201, 301) for at least two technologies (A, B), whereby an input by the planner interactively conveys the equivalence of two technologies to the same planner, and the two technologies (201, 301) shown have the same value (c1₁) for the same target criterion (c1) for the respective patch.

2. Method according to claim 1, whereby a resulting Pareto frontier is shown on the display device (10) as a patch (801) that tracks multiple sections of multiple Pareto frontiers shown as patches (101, 201, 401) of shown technologies (A, B, . . . Z) to make it easier for the planner to visibly navigate the resulting patch (801) across technologies to find a treatment plan.

3. Method according to claim 1, whereby the position of the selector of an operating aid is changed linearly or as a rotational position,
or
more than two target criteria (including plan targets) are shown on the display device (10) by means of projections in the patch area.

4. Method according to one of the preceding claims, whereby one of the target criteria is a treatment time.

5. Method according to one of the preceding claims, whereby the fuzziness interval (30, 31) is shown interactively by widening the selector (21a) of the operating aid (21) and is also represented in the patch area by an extended field (30′, 31′) for interactive communication with the planner, covering at least two Pareto frontiers (201, 301) as at least two patches.

6. Method according to one of claims 3 to 5, whereby the operating aid is linear or is an operating aid with a curved adjustment range.

7. Method according to one of the preceding claims, whereby the radiation device or devices are adapted to treat a person (P) with a tumor disease.

8. Method according to one of the preceding claims, whereby a window is displayed on the display device (10) as a function field (80) in which the planner marks selected technologies.

9. Method according to claim 8, whereby technologies corresponding to the Pareto frontiers (201, 202) are displayed as a function field (80) via the window shown in the operating area (1) for selection or deselection by the planner or are selected by the planner and also displayed or depicted in the operating aids (21, 22) when selected.

10. Method for designing or fashioning a treatment plan with interactive navigation on an input/output device, consisting of a display device (10).
(a) whereby multiple treatment technologies (A, B, . . . Z) are available for a planner to choose from, which are provided by multiple radiation devices (100, 200, 300, 400);
(b) whereby the designed or fashioned treatment plan defines a plurality of technical settings that are adjustable on the at least one radiation device (100, 200, 300) of the selected technology/technologies;
(c1) one Pareto frontier (101, 201, 301) each is displayed on the display device (10) of the input/output device for at least some of the technologies in a Pareto frontier diagram, and each of the Pareto frontiers is called a patch, whereby each of the Pareto frontiers is displayed as a function of at least two criteria (c1, c2) in a patch area (1) of the display device (10);
(c2) an operating area (2) outside the patch area (1) on the display device (10) shows a number of operating aids (21, 22, . . . ), especially as sliders, each operating aid of which represents a criterion (c1, c2, . . . ) and each of which operating aids has a selector (21a, 22a, . . . ) whereby a selector (22a) of the respective operating aid is improved when it is moved or operated in one direction or worsened when it is moved or operated in the opposite direction;
(c3) and, for interactive communication with the planner, each operating aid uses the associated selector to show a solution selected in the current state in the patch area (1) as a point zᵢ on one of the multiple Pareto frontiers, whereby the selected solution is changed along multiple patches;
(d) and, with a fuzziness interval (30, 31, 32) that is defined for a target criterion (c1, c2, . . . ) and that is also displayed accordingly in the interactive patch area and covers at least two Pareto frontiers (201, 301) as at least two treatment technologies, whereby an input by the planner interactively conveys the equivalence of two technologies to the same planner from the patch area (1), whereby the two treatment technologies shown have the same value (c1) for the same target criterion (c1) in the respective patch.

11. Method according to previous claim 10, whereby the selected solution according to section (c3) is changed along the multiple patches by the planner changing the position of a respective selector (21a) of a respective operating aid (21).

12. Method according to one of the preceding claims, whereby one change in position of the selector (21a) occurs
a. by changing the position of point (zᵢ) in patch area (1);
or
b. by changing the position of the selector (21a) of a respective operating aid (21) in the operating area (2);
whereby the point shown in the patch area (1) and all selectors in the operating area (2) are functionally coupled.

13. Method according to one of the previous claims, whereby the at least one radiation device (100, 200, 300, 400) involves at least one radiation device (100) for emitting protons or photons or for emitting accelerated heavy ions or for emitting accelerated electrons, or it is a brachy device (400) for emitting photons or gamma rays from advanced radiation bodies subject to radioactive decay.

14. Method according to previous claim 13, whereby the radiation bodies have a cylindrical shape with a length of less than 5 mm and consist of cobalt-60, iodine-125, or iridium-192, each preferably surrounded by a titanium shell.

15. Method according to one of previous claims 10 to 14, whereby a combined, global Pareto frontier (801) is formed from partial areas of the multiple Pareto frontiers; preferably, the selected solution is changed along the combined global Pareto frontier as a resulting Pareto frontier (801).

16. Method for designing or fashioning a treatment plan with interactive navigation on an input/output device, consisting of a display device (10),
(a) whereby multiple treatment technologies (A, B, . . . Z) are available for a planner to choose from, which are provided by at least one radiation device (100, 200, 300, 400);
(b) whereby the designed or fashioned treatment plan defines a plurality of technical settings that are adjustable on the at least one radiation device (100, 200, 300) of the selected technology/technologies;
(c1) one Pareto frontier each is displayed on the display device (10) of the input/output device for at least some of the technologies in a Pareto frontier diagram, and each of the Pareto frontiers is called a patch,
whereby each of the patches is displayed as a function of at least two criteria (c1, c2) in a patch area (1) of the display device (10);
(c2) an operating area (2) outside the patch area (1) on the display device (10) shows a number of operating aids (21, 22, . . . ), especially as sliders, each operating aid of which represents a criterion (c1, c2, . . . ) and each of which operating aids has a selector (21a, 22a, . . . ) whereby a selector (22a) of the respective operating aid is improved when it is moved or operated in one direction or worsened when it is moved or operated in the opposite direction;
(c3) and, in interactive communication with the planner, each operating aid uses the associated selector to show a solution selected in the current state in the patch area (1) as a point zᵢ on one of the multiple patches, whereby the selected solution is changed along at least two patches;
(d) with a fuzziness interval (30, 31, 32) that is defined for a target criterion (c1, c2, . . . ) and that is also displayed accordingly in the interactive patch area and covers at least two Pareto frontiers as at least two treatment technologies, whereby an input by the planner interactively conveys the equivalence of two technologies visually to the same planner from the patch area (1), whereby the two treatment technologies shown have the same value (c1₁) for the same target criterion (c1) in both patches.
(e) whereby a resulting Pareto frontier is shown on the display device (10) as a patch (801) that picks up multiple sections of multiple Pareto frontiers shown as patches (101, 201, 401) of shown technologies (A, B, . . . Z) to make it easier for the planner to visibly navigate the resulting patch (801) across technologies to find a treatment plan.

17. Method according to previous claim 16, whereby the selected solution according to section (c3) is changed along the at least one patch by the planner changing the position of a respective selector (21a) of a respective operating aid (21).

18. Method according to one of preceding claims 16 or 17, whereby one change in position of the selector (21a) occurs
a. by changing the position of point (zᵢ) in patch area (1);
or
b. by changing the position of the selector (21a) of a respective operating aid (21) in the operating area (2);
whereby the point shown in the patch area (1) and all selectors in the operating area (2) are functionally coupled.

19. Method according to one of previous claims 16 to 18, whereby the point (zᵢ) shown in the patch area (1) and all selectors in the operating area (2) are functionally coupled.

20. Technically usable GUI as a graphical-interactive interface for interactive operation by a planner with a function pointer (M) on or at a display device (10) for carrying out the method according to one of the preceding claims, whereby the technically usable GUI
(a1) displays one Pareto frontier (101, 201, 301) each on the display device (10) of the input/output device for at least some technologies in a Pareto frontier diagram, and each of the Pareto frontiers is called a patch, whereby each of the Pareto frontiers is displayed as a function of at least two criteria (c1, c2) in a patch area (1) of the display device (10);
(a2) shows a number of operating aids (21, 22, . . . ) in an operating area (2) outside the patch area (1) on the display device (10), each operating aid of which represents a criterion (c1, c2, . . . ) and each of which operating aids has a selector (21a, 22a, . . . ) whereby a selector (22a) of the respective operating aid can be improved when it is moved or operated in one direction or worsened when it is moved or operated in the opposite direction;
(a3) and, for interactive communication with the planner, uses each operating aid (21, 22, . . . ) with the associated selector (21a, 22a, . . . ) to show a solution selected in the current state in the patch area (1) as a point (zᵢ) on one of the multiple Pareto frontiers (101, 201, 301), whereby the selected solution can be changed along multiple patches;
(d) an fuzziness interval (30, 31, 32) is identifiable that is defined for a target criterion (c1, c2, . . . ) and that is also displayed by a highlighted strip accordingly in the interactive patch area and covers at least two Pareto frontiers (201, 301) as at least two treatment technologies with its extension (30″, 31″, 32″), whereby an input by the planner is able to interactively convey the equivalence of two technologies to the same planner from the patch area (1) in such a way that the two treatment technologies shown have the same value (c1) of the same target criterion (c1) in the respective patch.

21. GUI according to the preceding claim 20, whereby the fuzziness interval (30, 31, 32) is displayed in the interactive patch area in a vertically or horizontally oriented representation.

22. GUI according to one of preceding claims 20 or 21, whereby the fuzziness interval (30, 31, 32) is less than 10% points.

23. GUI according to one of preceding claims 20 to 22, whereby the selectors (21a, 22a) are functionally coupled with the associated points (zᵢ) on one of the plurality of Pareto frontiers.

24. GUI according to one of preceding claims 20 to 23, whereby technologies corresponding to the Pareto frontiers (201, 202) are displayed over a field (80) shown in the operating area (1) for selection or deselection by the planner, whereby they are displayed or depicted in the operating aids (21, 22) when selected.

25. Use of a functional pointer (M) for a graphical user interface for designing or fashioning a therapy as a treatment plan during interactive navigation by a planner as a user on a display device (10)
(a1) for at least some of the technologies (A, B, . . . Z), one Pareto frontier (101, 201, 301) each is shown connected on the display device (10), each of which is referred to as a patch;
(a2) a number of operating aids (21, 22, . . . ) are shown in an operating area on the display device (10), each of which represents one criterion (c1, c2, . . . ) that permits moving or operating a selector (21a, 22a, . . . ) of each operating aid;
(a3) and, for interactive communication with the planner, the respective selector (21a, 22a) highlights a solution selected in the current state in the patch area (1) as a point (zᵢ) on one of the multiple Pareto frontiers (101, 201, 301), and a selected solution is changed by the function pointer (M) along multiple patches;
(b) with a fuzziness interval (30, 31, 32) that is defined for a target criterion (c1, c2, . . . ) and that is also displayed accordingly in the interactive patch area and covers at least two Pareto frontiers (201, 301) for at least two technologies (A, B), whereby the equivalence of two technologies is interactively conveyed to the planner.

26. Use according to a claim 25, whereby the fuzziness interval (30, 31, 32) is displayed in the interactive patch area in a vertically or horizontally oriented representation.

27. Use according to one of preceding claims 25 or 26, whereby the fuzziness interval (30, 31, 32) is less than 10% points.

28. Use according to one of preceding claims 25 to 27, whereby the selectors (21a, 22a) are functionally coupled with the associated points (zᵢ) on one of the plurality of Pareto frontiers.

29. Use according to one of preceding claims 25 to 28, whereby technologies corresponding to the Pareto frontiers (201, 202) are displayed over a function field (80) shown in the operating area (1) for selection or deselection by the planner, whereby they are displayed or depicted in the operating aids (21, 22) when selected.
